(19) United States
(12) Patent Application Publication
(10) Pub. No.: US 2025/0312070 A1
(43) Pub. Date: Oct. 9, 2025

(54) DOUBLE TULIP SPINAL FIXATION CONNECTORS AND RELATED METHODS AND APPARATUS

(71) Applicant: Ortho Development Corporation, Draper, UT (US)

(72) Inventors: David F. Waller, Draper, UT (US); Bao-Khang Ngoc Nguyen, Salt Lake City, UT (US); Stephen Paul Brown, Cedar City, UT (US)

(21) Appl. No.: 19/090,301

(22) Filed: Mar. 25, 2025

Related U.S. Application Data
(60) Provisional application No. 63/575,369, filed on Apr. 5, 2024.

(51) Int. Cl. A61B 17/70

(52) U.S. Cl. CPC A61B 17/7034; A61B 17/7035

(57) ABSTRACT

Spinal fixation coupling devices and systems, such as tulip connectors having extending arms and/or coupling heads extending therefrom to form double tulip connections, and related apparatus, methods, and systems. In some embodiments, a primary tulip connector configured to couple both a spinal fixation rod and a bone screw may further comprise a coupling head projecting therefrom, in some cases terminating at the end of an extending arm. The coupling head may be configured to allow for coupling of a secondary tulip connector to the primary tulip connector to facilitate coupling to a second rod. In some embodiments, the secondary tulip connector may be rotatable and/or adjustable, such as adjustable in roll, pitch, and/or yaw, with respect to the coupling head and/or one or more portions of the primary tulip connector.

### CROSS-REFERENCE TO RELATED APPLICATIONS

This application claims the benefit under 35 U.S.C. § 119(e) of U.S. Provisional Patent Application No. 63/575,369, which was filed Apr. 5, 2024, and titled “DOUBLE TULIP SPINAL FIXATION CONNECTORS AND RELATED METHODS AND APPARATUS,” which is hereby incorporated herein by reference in its entirety.

### BACKGROUND

Spinal correction surgeries are often used to relieve pain and restore a person's mobility and quality of life. Surgical correction of the human spine is complex due to the multi-joined, curved nature of the spine, with boney vertebrae segmented with flexible discs allowing the body and head to move in complex motions. Each motion segment anatomically can vary greatly as needed for each level including muscle attachments, boney attachments, nervous system routing, and physiologic loading. Deformities, trauma, osteoporosis, stenosis, and injuries can limit a person's ability to move freely and/or cause great pain.

Spinal rod systems are one method of correcting/stabilizing spinal deformity, by bracing the injured motion segment with spinal rods. Basic elements of spinal rod systems generally include bone screws to anchor the vertebrae and tulips which connect bone screws to spinal rods, which function as the brace. Additionally, other deformity correction devices can provide surgeons with options for avoiding poor bone that cannot hold a bone screw well and for adding additional strength to the construct to correct the deformity or other correction methods.

### SUMMARY

Disclosed herein are various embodiments of spinal deformity correction apparatus and systems, along with related methods.

In some cases, the surgeon determines the need for multiple rods to provide greater stiffness and/or strength in a particular region of the spine. Some embodiments and implementations disclosed herein provide devices, systems, and/or means for a surgeon to connect a single bone screw to two or more spinal rods and/or for cross-linking multiple rods to create a more stable bracing construct. In some cases, tulips or tulip-like devices may be used that connect to a bone screw and to a single rod. Some embodiments may further comprise a coupling head, which may be configured to couple with a second tulip or other similar device, such as a rod clamp or a tether clamp, and thereby couple the assembly/system to a second rod or boney structure. In some cases, the coupling head may be attached, in some cases integrally attached, to an extended or projecting member that may be used to position the coupling head at a desired location that may be spaced apart from a primary connector.

In some embodiments, the primary tulip or other connector—which may be configured to receive both a screw, such as a pedicle screw, and a spinal fixation rod—may also include a coupling member, such as the aforementioned coupling head, projecting from a portion thereof. In some cases, a post and/or a coupling head may project from the primary connector, which coupling head may be configured to be received in a secondary coupling member, such as another tulip. In some embodiments, the coupling head may comprise a spherical, frusto-spherical, or at least substantially spherical coupling head.

The secondary connector may then be coupled with the primary tulip/connector. In some cases, a standard tulip that is configured to be mounted onto a pedicle screw may be mounted/coupled onto the coupling head of the primary coupling member. In some cases, the secondary coupling member may therefore comprise a tulip connector with no, or at least substantially no, changes from a tulip connector configured to receive the head of a typical pedicle screw. The projecting coupling member and/or spherical coupling head may therefore be configured, in some embodiments to mimic or at least substantially mimic the head of a pedicle screw. Of course, other embodiments are contemplated, some of which are disclosed herein, in which the configuration of the projecting coupling member and/or spherical coupling head may be modified, which may therefore warrant changes to the corresponding secondary coupling member/tulip, if needed.

In one aspect of this disclosure, the first/primary tulip/connector may be configured to be adjustable relative to the spinal rod coupled therewith. For example, in some cases, the first/primary tulip may be configured to be adjustable in “yaw” relative to the spinal rod coupled therewith, in some cases within a predetermined angle range. In some such cases, the first/primary tulip may be configured to allow for rotation in all directions (i.e., a polyaxial coupling) relative to the spinal rod coupled therewith. This may provide for further adjustability and/or flexibility for the surgeon to solve challenging problems.

In some cases, the first/primary tulip may be configured to be fixed in translation relative to the secondary coupling member. In other words, in some embodiments, the secondary coupling member, once mounted on the projecting arm or other suitable coupling member, may be fixed other than the aforementioned rotational adjustment such that the spacing between the two connectors is fixed, or at least substantially fixed.

In another aspect of this disclosure, the connection to other tulips and/or connectors may be by means of an extending arm or other projecting member from the first tulip body to the second tulip body that terminates in a spherical, or at least substantially spherical, ball or other coupling head. This coupling head may be, for example, parallel to the extending arm or perpendicular to the extending arm. The extending arm may be perpendicular to the side of the first tulip or angled downward or distally.

In another aspect of this disclosure, the first tulip/connector may have two opposed extending arms or projecting members, each of which terminates in a coupling head, such as a spherical or at least substantially spherical ball. In this embodiment, the device can be connected to a bone screw and to three spinal rods. In some cases, the coupling head or other coupling member may comprise features to facilitate locking of the secondary connector at a desired orientation, such as facets and, in some cases, corresponding coupling elements having similar facets, such as locking rings and/or saddles.

In each of these aspects, the secondary connector or second tulip may be any tulip-like device that is configured to attach to the coupling head, such as a spherical head in some cases, of a bone screw. Thus, it may be preferred that the coupling head on the distal end of the extending arm be the same or similar in size and/or shape as the head on the proximal end of the bone screw. This allows the surgeon the option of attaching a standard tulip, a raised height tulip, or an offset tulip, or any other device that can connect to a second rod.

In another aspect of this disclosure, the coupling head on the distal end of the extending arm may be larger or smaller than the head on the proximal end of the bone screw. This may limit the second tulip body to only those that are specifically designed for use in connection with a particular system rather than standard tulip bodies usable with a wide range of pedicle screws.

In some embodiments, the attachment of the second tulip/connector to the coupling head on the distal end of the extending arm provides the ability of the tulip and the second spinal rod to be adjusted in yaw, pitch, and roll in relation to the first tulip and the first spinal rod.

In some embodiments, the connection of the bone screw to the first or primary tulip may be limited to allow adjustment in only one or two directions. Likewise, the connection of the second/secondary tulip or similar device to the ball or other coupling head on the end of the extending arm may be limited to allow adjustment in only one or two directions in some cases. The limitation on the bone screw connection and on the second tulip connection may be independent and may therefore be independently selected as needed for a particular application.

In another aspect, the secondary tulip body or similar device may be configured to be rotatable relative to the first/primary tulip/connector such that the spinal rod slot of the secondary tulip/connector is angled rather than parallel, or in some cases perpendicular or at least substantially perpendicular, to the first spinal rod. In some such embodiments, the system may be configured to accommodate a transverse connection from the spinal rod construct on one side of the spine to the spinal rod construct on the other side of the spine. In addition to, or as an alternative to, providing for such rotation, some embodiments may further allow the secondary tulip/connector body to be pivoted in one or more planes relative to the primary tulip/connector and/or one or more components thereof.

In another example of a spinal connector device according to some embodiments, the device may comprise a body, which body may comprise an opening formed in a distal end thereof. The opening may be configured to receive a portion of a fastener, such as the head of a pedicle or other bone screw, therein. A slotted opening may be formed along a proximal portion of the body, which may be configured to receive a spinal fixation rod or other rod therein. An extending arm may extend from and/or be coupled to the body. In some cases, the extending arm may integrally extend from the body. A coupling member, such as a coupling head, may be coupled and/or extend from, in some cases integrally therefrom, the extending arm. The coupling member may be configured to be received within the body of a separate spinal connector device.

In some embodiments, the coupling member may comprise a spherical or at least substantially spherical coupling head. In some embodiments, including but not limited to some such embodiments, the coupling member may be configured to mimic the head of another fastener, such as a pedicle screw head. In some such embodiments, the coupling member/head may be configured to mimic the head of the screw or other fastener that is configured to be received in the opening of the body itself.

In some embodiments, the coupling member may define an at least substantially cylindrical lateral surface, which may allow for rotation of the separate spinal connector device without allowing for pivoting and/or tilting thereof relative to the connector device.

In some embodiments, the extending arm may extend below a lower end of the body defining the opening.

Some embodiments may further comprise a second extending arm, which may extend, in some cases integrally, from the body. In some such embodiments, a second coupling member may extend from, in some cases integrally from, the second extending arm. The second coupling member may be configured to be received within the body of another, separate spinal connector device. In some embodiments, the second extending arm may extend from an opposite surface of the body relative to the extending arm. For example, in some such embodiments, the two extending arms may each begin from locations connectable by a straight line extending through the body, in some cases a straight line perpendicular to a central axis of the body corresponding to the opening for the bone screw below.

In another example of a spinal tulip connector system according to some embodiments, the system may comprise a first tulip body comprising a slotted proximal opening configured to receive a spinal fixation rod therein and a distal opening configured to receive a screw head or other fastener head therein. A coupling head may be coupled with, in some cases integrally coupled with, the tulip body. The system may further comprise a second tulip body comprising a distal opening configured to receive the coupling head therein.

Some embodiments may further comprise an extending arm extending from, in some cases integrally extending from, the tulip body. The coupling head may then extend from, in some cases integrally from, the extending arm.

In some embodiments, the coupling head may extend at a perpendicular angle, in some cases at an at least substantially perpendicular angle, from the extending arm.

Some embodiments may further comprise a bone screw comprising a bone screw head. The bone screw head may be configured to be received in the distal opening of the first tulip body. In some such embodiments, the coupling head may comprise a shape and/or size that matches, or at least substantially matches, a shape and/or size of the bone screw head.

In some embodiments, the coupling head may comprise a plurality of facets configured to facilitate locking of the second tulip body at a desired orientation relative to the coupling head and/or first tulip body.

In another example of a spinal connector system according to some embodiments, the system may comprise a primary connector, such as a tulip connector, which may comprise a body defining a slotted opening configured to receive a rod therein. The primary connector may further comprise an opening formed in a lower portion of the body, the opening configured to receive a portion of a bone fastener therein. The primary connector may further comprise an extending arm extending from, in some cases integrally from, an outer surface of the body. A coupling member, such as a coupling head, may extend from, in some cases integrally from, the extending arm. The system may further comprise a secondary connector, which may comprise a body and an opening formed in a lower portion of the body. The opening may be configured to receive the coupling member therein to secure the primary connector to the secondary connector.

In some embodiments, the primary connector and/or the secondary connector may comprise a tulip connector.

In some embodiments, the secondary connector may further comprise a proximal opening positioned above the opening. The proximal opening may be configured to receive a saddle and/or a set screw for securing the coupling member within the secondary connector. In some embodiments, the secondary connector may further comprise a secondary body portion, which may extend from and/or be laterally offset from the body. In some cases, the secondary body portion may comprise a slotted opening configured to receive a rod therein such that the rod for the secondary connector may not only be offset from a rod coupled with the primary connector, but also offset from the coupling member to any desired degree.

In some embodiments, the secondary connector may be configured to be adjustable relative to the primary connector in roll, pitch, and/or yaw.

The features, structures, steps, or characteristics disclosed herein in connection with one embodiment may be combined in any suitable manner in one or more alternative embodiments.

### BRIEF DESCRIPTION OF THE DRAWINGS

Non-limiting and non-exhaustive embodiments of the disclosure are described, including various embodiments of the disclosure with reference to the figures, in which:

FIG. 1 is a perspective view of a first embodiment of a primary tulip connector comprising a spherical coupling head extending from the side thereof;

FIG. 2 is a perspective view of a second embodiment of a primary tulip connector comprising a projecting arm terminating at a spherical coupling head;

FIG. 3 is a perspective view of a third embodiment of a primary tulip connector comprising a downwardly extending projecting arm terminating at a spherical coupling head;

FIG. 4 is a perspective view of a fifth embodiment of a primary tulip connector comprising two separate projecting arms each terminating at its own spherical coupling head;

FIG. 5 is a cross-sectional view of a primary tulip connector shown coupled with a secondary tulip connector coupled to the coupling head of the primary tulip connector;

FIG. 6 is an exploded view of a primary tulip connector having a faceted coupling head and a series of elements configured to facilitate coupling to a secondary tulip connector;

FIG. 7 is an exploded view of another primary tulip connector having a post- like coupling head and a series of elements configured to facilitate coupling to a secondary tulip connector;

FIG. 8 is a perspective view of a spinal fixation system comprising two primary tulip connectors each coupled with its own secondary tulip connector, wherein both of the tulip connectors are shown receiving parallel spinal fixation rods to define a spinal fixation system;

FIG. 9 is a top plan view illustrating how, in some embodiments, the primary tulip connector can twist or yaw in relation to the spinal rod over a limited range;

FIG. 10 is a top plan view illustrating how the ability to adjust the yaw angle of the primary tulip connector can be used to adjust the angle of the secondary tulip connector as needed;

FIG. 11 is a perspective view of another spinal fixation system in which two parallel spinal fixation rods are coupled by a pair of primary/secondary tulip connectors and incorporating an inline connector to include a third spinal fixation rod aligned with one of the first two;

FIG. 12 is a perspective view of yet another spinal fixation system including two parallel spinal fixation rods and two transverse spinal fixation rods connected by a pair of primary and secondary tulip connectors;

FIG. 13 is a perspective view of a spinal fixation system comprising primary connector having a projecting arm configured to be secured within an opening formed in a secondary connector;

FIG. 14 illustrates the spinal fixation system of FIG. 13 following securement of the primary connector vis-à-vis the secondary connector and with a spinal fixation rod secured within a slotted opening of each connector; and

FIG. 15 is a perspective view illustrating the lower surface of the saddle of the secondary connector of the embodiment of FIGS. 13 and 14 that is configured to engage the projecting arm of the primary connector.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

FIG. 1 depicts a primary tulip connector 100 comprising a spherical coupling head 108 extending from the side thereof. More particularly, spherical coupling head 108 extends from a base 107 that extends from a lower portion of the connector 100. As will be apparent following the discussion below, this may allow for coupling of two spinal fixation rods or other similar fixation members in a parallel, or at least substantially parallel, manner. Those of ordinary skill in the art will appreciate, however, that base 107 and coupling member 108 may extend from any portion of the tulip body of connector 100, as desired.

Primary tulip connector 100 comprises a distal end 101 and a proximal end 102. An opening 103 is formed in the distal end 101 for receiving the proximal end, such as the head, of a bone screw or other fastener. A slotted opening 104 extends downward from the proximal end 102 for receiving a spinal rod (not shown). The slotted opening 104 may comprise threads 105 that may be configured to engage a threaded blocker or cap, such as a set screw (not shown).

The body of the connector 100 has a centerline CL11 that extends from the proximal end 102 to the distal end 101. The spherical coupling member 108 also has a centerline CL12 that extends from the center of the coupling member 108 through the body of the connector 100 transversely to the body centerline CL11. In the depicted embodiment, centerline CL12 is perpendicular, or at least substantially perpendicular, to the centerline CL11. However, as discussed below in connection with other embodiments, this need not always be the case.

A secondary connector (not shown) may be coupled with the coupling member 108. In some embodiments, coupling member 108 may be configured to mimic or resemble the head of an existing bone screw. Thus, in some cases, an existing tulip configured to engage such a bone screw may be used without modification, or at least substantially without modification, by simply coupling such a tulip/connector to the spherical coupling member 108. In this manner, various embodiments disclosed herein may be configured to operate in conjunction with known and/or preexisting bone fixation system elements.

Because of the spherical or at least substantially spherical shape of coupling member 108, some embodiments may be configured to allow the secondary connector to pivot and/or rotate about the spherical coupling member 108 in order to receive a spinal rod or other elongated fixation element at different positions and/or angles relative to the rod/element received in slotted opening 104.

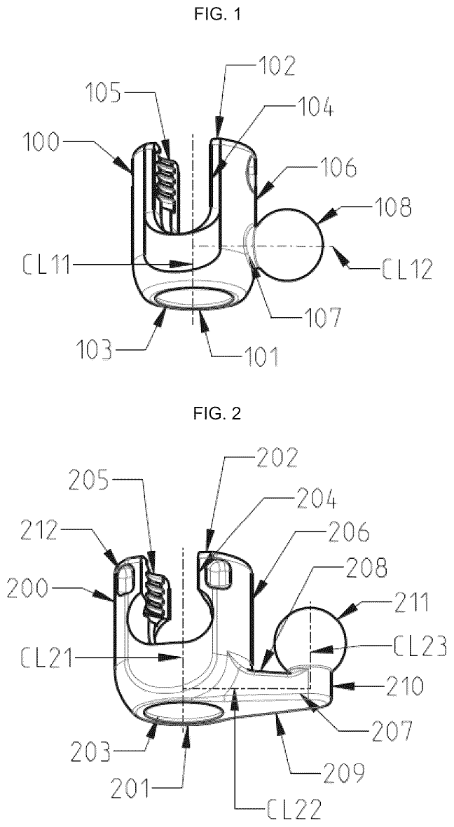

FIG. 2 is a perspective view of another primary tulip connector 200 according to other embodiments. Like connector 100, connector 200 comprises a tulip body comprising a distal end 201 and a proximal end 202. An opening 203 is formed in the distal end 201 for receiving the proximal end, such as the head, of a bone screw or other fastener. A slotted opening 204 extends downward from the proximal end 202 for receiving a spinal rod (not shown). The slotted opening 204 may comprise threads 205 that may be configured to engage a threaded blocker or cap, such as a set screw (not shown).

Unlike connector 100, however, connector 200 comprises a widened portion in the slotted opening 204. This widened portion may allow the rod receiving in slotted opening 204 to be pivoted in either direction to selectively change the angle of the rod within a predetermined angle range. In some embodiments, this angle range may allow for rotation of the rod within the slotted opening 204 within an angle range anywhere between about ±2 degrees and ±45 degrees from the axial or neutral configuration. In some embodiments, this angle range may be more limited, which may be preferable, for example, due to the weakening of the tulip resulting from the widened portion. Thus, in some embodiments, the angle range may only allow for rotation of the rod at any angle up to about ±20 degrees. In some such embodiments, the angle range may only allow for rotation of the rod at any angle up to about ±10 degrees or, in other cases, any angle up to about ±5 degrees.

Detents 212 are also formed along the upper rim of the tulip body of connector 200. These detents 212 may be configured to provide a means for grasping the connector 200 with an instrument. In the depicted embodiment, two detents 212 are shown, but it should be understood that two additional detents may be positioned on the opposite side and fewer or more such detents (including no detents at all) may be used in other embodiments.

The tulip body again has a centerline CL21 that extends from the proximal end 202 to the distal end 201. An extending arm 207 protrudes from the body and terminates in a spherical coupling member 211. In the depicted embodiment, coupling member 211 extends upwards and is configured to receive a secondary tulip and/or coupling member in a manner that allows for the rod received therein to extend parallel to, and at the same, or substantially the same, height as the rod extending through slotted opening 204. However, again, this need not be the case for all contemplated embodiments. Indeed, as will be more apparent after considering other embodiments discussed below, the height of the secondary connector, along with the corresponding height and angle(s)—including in some cases the yaw, pitch, and/or roll—of the rod received in the secondary connector, may differ and may, in some embodiments, be adjustable.

As but one example before discussing other embodiments, it is contemplated that the coupling member 211 of connector 200 may protrude laterally, or even downward, rather than upward as shown in FIG. 2. In addition, although the shape of the coupling member 211 may preferably be spherical, as shown in this figure, for some applications, this shape may also vary as desired.

Extending arm 207 is formed by a proximal surface 208 that extends from a side 206 of the body of the tulip and a distal surface 209 that extends from the distal end 201 of the body and terminates at an end surface 210. Again, however, alternative embodiments are contemplated. For example, the lower portion of the extending arm 207 may, similar to proximal surface 208, extend from a side of the body of the connector 200 instead of the distal end 201.

The extending arm 207 has centerline CL22 parallel, or at least substantially parallel, to the proximal surface 208 of the extending arm 207. In this embodiment, the proximal surface 208 of the extending arm 207 is also perpendicular, or at least substantially perpendicular, to the side 206 of the body. The end surface 210 of the extending arm 207 protrudes proximally, or upward, from the proximal surface 208, terminating in a spherical ball 211. The end surface 210 and the spherical coupling head/ball 211 both have a centerline (centerline CL23 of coupling head 211, for example) that is perpendicular, or at least substantially perpendicular, to the centerline CL22 of the extending arm 207.

As previously mentioned, spherical coupling head 211 may be configured to receive a secondary connector (not shown) and may be configured to mimic or resemble the head of an existing bone screw to allow for the use of an existing tulip configured to engage such a bone screw without, or at least substantially without, modification.

In addition, because of the spherical or at least substantially spherical shape of coupling head 211, some embodiments may be configured to allow the secondary connector to pivot and/or rotate about the spherical coupling head 211 in order to receive a spinal rod or other elongated fixation element at different positions and/or angles relative to the rod/element received in slotted opening 204.

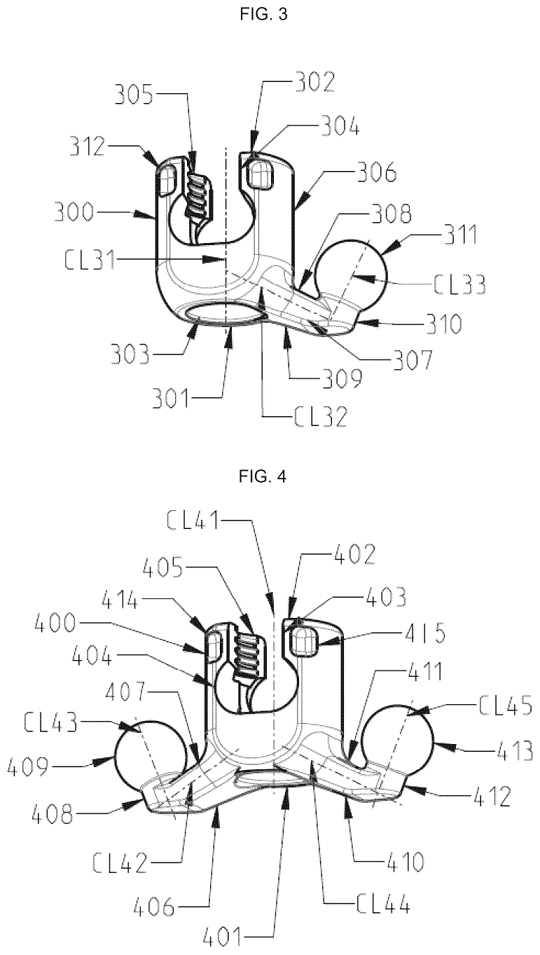

FIG. 3 is a perspective view of a primary tulip connector 300 according to further embodiments. Connector 300, unlike connectors 100 and 200, comprises a downwardly projecting extending arm 307, which, like connector 200, also terminates at a spherical coupling head 311.

Again, connector 300 comprises a tulip body comprising a distal end 301 and a proximal end 302. An opening 303 is formed in the distal end 301 for receiving the proximal end, such as the head, of a bone screw or other fastener. A slotted opening 304 extends downward from the proximal end 302 for receiving a spinal rod (not shown). The slotted opening 304 may comprise threads 305 that may be configured to engage a threaded blocker or cap, such as a set screw (not shown). Slotted opening 304 comprises a widened portion that may allow the rod receiving in slotted opening 304 to be pivoted within a predetermined angle range, as previously mentioned. Of course, this may be omitted, if desired. For example, the slotted opening 304 may resemble that of slotted opening 104 of connector 100, or any other slotted opening available to those of ordinary skill in the art. Detents 312 may also be formed along the upper rim of the tulip body of connector 300 to allow for grasping the connector 300 with an instrument.

The tulip body again has a centerline CL31 that extends from the proximal end 302 to the distal end 301. Extending arm 307 protrudes from the body and terminates in a spherical coupling member 311. However, in this case, extending arm 307 protrudes at a downward angle. In some embodiments, this angle may be between about 5 and about 45 degrees or, in some such embodiments, between about 20 and about 45 degrees. Alternatively, the angle range may be between about 5 and about 20 degrees in some embodiments, or in other embodiments between about 20 and about 30 degrees. Moreover, this downward angle is but an example. It should therefore be understood that extending arm 307 may instead be angled upwards, or laterally to either side, as desired, including at any of the aforementioned angle ranges.

Coupling head 311 again extends upwards relative to arm 307 (but angled downward relative to the body of the connector 300 due to the downward angle of the arm 307) and is configured to receive a secondary tulip and/or coupling member in a manner that allows for the rod received therein to extend parallel to but at a lower height relative to the rod extending through slotted opening 304.

Extending arm 307 may, once again, be formed by a proximal surface 308 that extends from a side 306 of the body of the tulip and a distal surface 309 that extends from the distal end 301 of the body and terminates at an end surface 310. Again, however, the lower portion of the extending arm 307 may extend from other locations, such as a side of the body of the connector 300 instead of the distal end 301 in alternative embodiments.

The extending arm 307 has centerline CL32 that extends laterally and distally or downward relative to the side of the tulip body. In this embodiment, the proximal surface 308 of the extending arm 307 is therefore also angled downwardly relative to the side 306 of the body. The terminating surface 310 of the extending arm 307 protrudes at a perpendicular, or at least substantially perpendicular, angle from the proximal surface 308, terminating in a spherical coupling ball 311. The terminating surface 310 and the spherical ball 311 therefore again have a centerline CL33 that is perpendicular, or at least substantially perpendicular, to the centerline CL32 of the extending arm 307.

Spherical coupling head/ball 311 may, as previously described, be configured to receive a secondary connector (not shown) and may, in some cases, be configured to mimic or resemble the head of an existing bone screw to allow for the use of an existing tulip configured to engage such a bone screw without, or at least substantially without, modification. Of course, in other cases, any of the embodiments described herein, including but not limited to the embodiment of FIG. 3, may have other coupling heads that may not be spherical and/or may require specific secondary connectors rather than known/existing tulips/connectors.

FIG. 4 is a perspective view of a primary tulip connector 400 according to still other embodiments. Connector 400 comprises two separate projecting arms 406 and 410, each of which terminates at its own spherical coupling head 409/413.

Otherwise, connector 400 may be similar to those discussed above. For example, connector 400 comprises a tulip body comprising a distal end 401 and a proximal end 402. An opening 403 is formed in the distal end 401 for receiving the proximal end of a bone screw or other fastener. A slotted opening 404 extends downward from the proximal end 402 for receiving a spinal rod (not shown). The slotted opening 404 may comprise threads 405 that may be configured to engage a threaded set screw or the like. Slotted opening 404 may also comprise a widened portion, as previously mentioned. Detents 412 may also be formed along the upper rim of the tulip body of connector 400 to allow for grasping the connector 400 with an instrument, as also previously described.

The tulip body again has a centerline CL41 that extends from the proximal end 402 to the distal end 401. Extending arms 406 and 410 each protrude from the body from opposing sides thereof and each terminates in its own respective spherical coupling head 409/413. Both extending arms 406/410 protrude at a downward angle, which angle may be within any of the previously mentioned angle ranges. It should also be understood that, although this angle is the same for both arms 406/410, it is contemplated that, in other embodiments, these angles may differ. For example, one of the arms 406/410 may protrude more steeply than the other. Similarly, one of the arms 406/410 may protrude upward and the other one downward, if desired, or one of the arms may protrude laterally at a perpendicular, or at least substantially perpendicular, angle relative to the body of the connector 400 rather than up or down.

Both coupling heads 409/413 extend upwards relative to its respective arm 406/410 (but are angled relative to the body of the connector 400 due to the downward angle of the arms 406/410) and both are configured to receive a respective secondary tulip and/or coupling member/connector in a manner that allows for the rod received therein to extend parallel to but at a lower height relative to the rod extending through slotted opening 404.

Extending arms 406/410 are each formed by a respective proximal surface 407/411 that extends from respective, opposing sides of the body of the tulip and both have respective distal surfaces that extend from the distal end 401 of the body and terminate at respective end surfaces 408/412. Again, however, the lower portion of the extending arms 406/410 may extend from other locations, such as a side of the body of the connector 400 instead of the distal end 401 in alternative embodiments.

Each of the extending arms 406/410 has its own respective centerline CL42/CL44 that extend laterally and distally or downward relative to the tulip body of the connector 400. In this embodiment, the proximal surfaces 407/411 of the extending arms 406/410 are therefore also angled downwardly relative to the side of the body. The terminating surfaces 408/412 of the extending arms 406/410 also protrude at a perpendicular, or at least substantially perpendicular, angle relative to the proximal surfaces of the arms 406/410, both of which terminating at a respective spherical coupling ball 409/413 or other coupling head. The terminating surfaces 408/412 and the coupling heads 409/413 therefore each has its own centerline (CL43/CL45 for the coupling heads 409/413) that is perpendicular, or at least substantially perpendicular, to the centerline CL42/CL44 of its respective extending arm 406/410.

Again, spherical coupling heads 409/413 may be configured to receive a respective secondary connector (not shown) and may, in some cases, be configured to mimic or resemble the head of an existing bone screw to allow for the use of an existing tulip configured to engage such a bone screw without, or at least substantially without, modification.

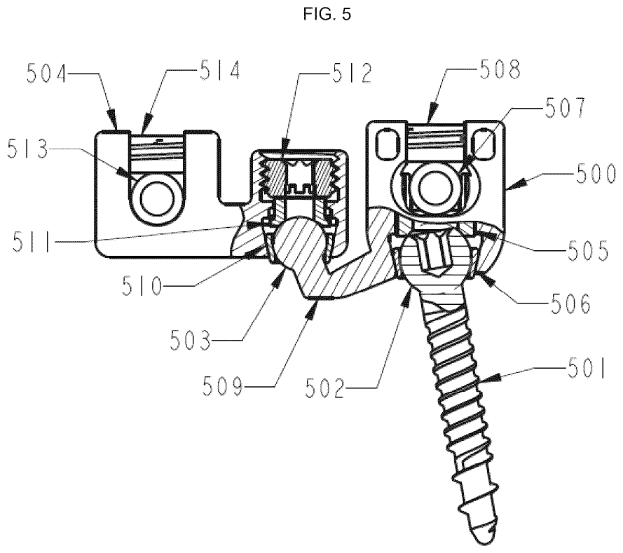

FIG. 5 is a cross-sectional view of a spinal fixation system including a primary connector 500 and a secondary connector 504 coupled to the coupling head of the primary connector 500. The primary connector 500 may be similar or identical to connector 300, given that it has a downwardly projecting extending arm and a coupling head 503 extending upwardly therefrom.

The primary connector 500 is shown receiving the head 502 of a bone screw 501. A ring 506 is shown within a lower chamber of the primary connector 500, which may facilitate a polyaxial coupling between the head 502 and the connector 500. In some embodiments, ring 506 may comprise a split-ring configured to expand and contract about the head 502 of screw 501.

An upper chamber of the primary connector 500 comprises a saddle 505, which is configured to engage both the upper end of the head 502 of screw 501 and the primary rod 507. A cap 508, which may comprise a threaded cap, may be used to lock the rod 507 and/or screw 501 in place. In some embodiments, locking of cap 508 may result in compression of rod 507 against saddle 505, which may, in turn, compress saddle 505 against screw head 502 to lock the screw 501 at its current angular configuration.

The secondary connector 504 comprises a coupling head receiving opening, which may be configured to receive and engage coupling head 503 in a manner similar to that of the lower chamber of primary connector 500 and/or the other secondary connectors previously mentioned. The extending arm 509 of primary connector 500 is configured to extend at a downward angle from the body of the connector 500 and has an upwardly extending and spherical coupling head 503.

It can also be seen in this figure that the coupling head 503 has a smaller size than that of the screw head 502. This may allow for the use of custom, proprietary, and/or different coupling assembly configurations. As alluded to above, it should be understood that this concept may be applied to any of the embodiments discussed herein. For example, embodiments having faceted or non-spherical coupling heads may be made of any shape and size as desired. Likewise, the embodiment of FIG. 5 may alternatively be configured with the coupling head 503 having the same, or at least substantially the same, size and/or shape as the screw head 502. This ability to utilize pre-existing and/or standard tulip connectors with the coupling heads may also be applied to any of the embodiments discussed herein.

Like the lower chamber of primary connector 500, the lower chamber of the secondary connector 504 comprises a ring 510, which may again comprise a split ring in some embodiments, for facilitating engagement with coupling head 503. This may allow the coupling head 503 to pivot within this chamber similar to the manner in which head 502 pivots within the lower chamber of primary connector 500.

However, rather than having a slot thereabove for receiving a spinal fixation rod or other similar fixation member, secondary connector 504 comprises a saddle 511 and a locking cap 512. These elements may be configured to fix the angle of the secondary connector 504 relative to the coupling head 503 at a desired orientation similar to the manner in which saddle 505 and cap 508 provide this function in primary connector 500. However, in the case of secondary connector 504, because there is no spinal rod present in this portion of the connector 504, the saddle 511 is configured to engage both the coupling head 503 and the locking cap 512 simultaneously.

Secondary connector 504 further comprises another slot spaced apart from the aforementioned slot for receipt of coupling head 503. This second slot is provided for receipt of a second spinal fixation rod 513 therein. Another locking cap 514 may be provided for locking this second rod 513 in place. By providing this configuration, two adjacent spinal rods can be coupled and fixed together at a variety of different heights and/or angles relative to one another.

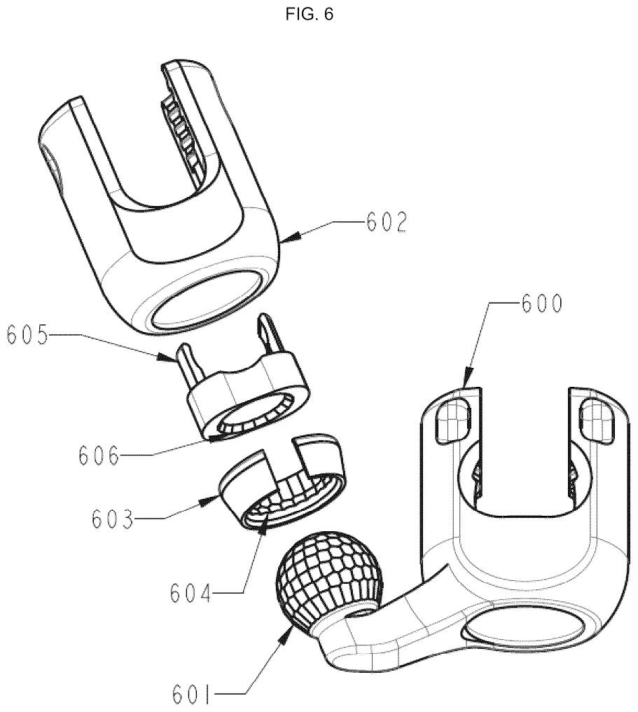

FIG. 6 is an exploded view of a system including a primary tulip connector 600 and a secondary tulip connector 602 according to further embodiments. Connector 600 comprises a faceted coupling head 601 that is fixedly and/or integrally coupled with an extending arm. By providing a plurality of flattened surfaces or facets along the spherical-shaped coupling head 601, restriction of secondary connector 602 to a limited number of specific rotational and/or pivotal orientations may be facilitated. This may also improve the ability to lock the secondary connector 602 in one of these orientations by increasing the force required to dislodge the secondary connector 602 from this configuration.

In the depicted embodiment, a split ring 603 is provided, which may be configured to flex open to allow for insertion of the coupling head 601 therethrough. Split ring 603 further comprises a plurality of facets 604 formed along the inner surface of the ring. Each of these facets 604 is configured to engage with a corresponding facet formed on coupling head 601 in order to lock the secondary connector 602 in place at a particular rotational orientation. Secondary connector 602 may have a tapered lower chamber that can receive ring 603 and compress ring 603 against coupling head 601.

The upper portion of the chamber, or in some cases a separate chamber above the aforementioned tapered lower chamber, may be configured to receive a saddle 605. Saddle 605 may comprise a pair of opposing posts or sidewalls that are configured to receive and seat a spinal fixation rod or other fixation member therein. The body of the saddle 605 may define a lower opening 606 within which an upper portion of the coupling head 601 may be received and/or engaged/seated. In the depicted embodiment, a portion of the inner surface defining this lower opening 606 may also have a plurality of facets formed thereon. This option may further enhance the locking stability of the secondary connector 602 on the coupling head 601 in a desired orientation.

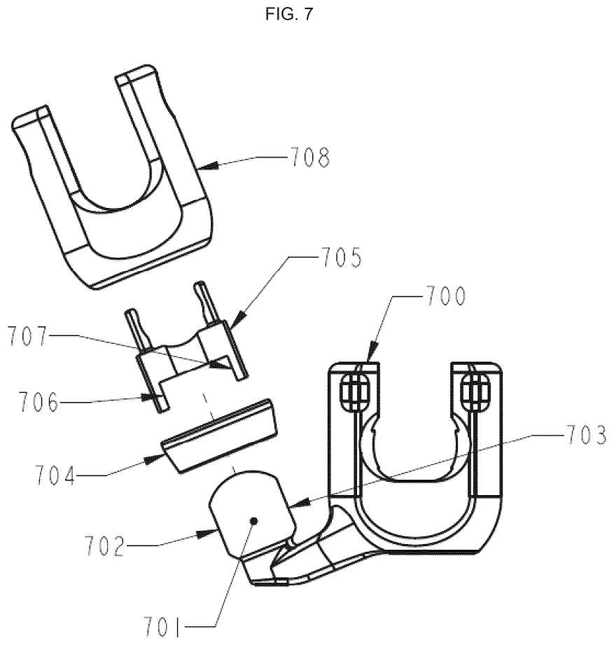

FIG. 7 is an exploded view of another system comprising a primary tulip connector 700 configured to couple with a secondary tulip connector 708. In this embodiment, primary connector 700 comprises a post-like or cylindrical coupling head 703. Like the system of FIG. 6, a series of coupling elements are provided, which are configured to facilitate coupling of the primary connector 700 to the secondary connector 708.

Connector 700 comprises a coupling head 703 that is fixedly and/or integrally coupled with an extending arm. The extending arm extends at a downward angle from the body of the primary connector 700. However, as should be apparent, this is but an example and the extending arm may extend straight out laterally, upwards, or in some cases may even be absent. In other words, the coupling head 703 may instead extend straight out from the body of the connector 700 without providing an extending arm.

As shown in FIG. 7, the coupling head 703 comprises flattened side surfaces 702 that may define a cylinder or an at least substantially cylindrical lateral surface. The upper portion of the coupling head 703 may, as shown in the figure, comprise a rounded dome-like tip. Alternatively, the upper portion may be flat in other embodiments.

The coupling head 703 may define an axis 701 that extends perpendicular, or at least substantially perpendicular, relative to the axis of the extending arm.

In the depicted embodiment, a ring 704 is provided. Because of the cylindrical rather than spherical shape of the coupling head 703, ring 704 need not be split to facilitate expansion and allow for insertion of the coupling head 703 therethrough. Secondary connector 708 may have a lower chamber that can receive ring 704 and facilitate mounting against coupling head 703.

The upper portion of the chamber, or in some cases a separate chamber above the aforementioned lower chamber, may again be configured to receive a saddle 705. Saddle 705 may comprise a pair of opposing posts or sidewalls that are configured to receive and seat a spinal fixation rod or other fixation member therein, in some cases using a snap-fit connection. The lower portion of the saddle 705 may similarly define a pair of downwardly projecting arms 706/707. These arms 706/707 may facilitate the seating/engagement of the ring 704 and the coupling head 703 within the secondary connector 708. More particularly, the arms 706/707 are configured to constrain the saddle 705 to the flattened side surfaces 702 on the coupling head 703, thereby creating a monopolar connection. In addition, by providing the aforementioned flattened side surfaces 702, the secondary connector 708 may be constrained to rotation within a single plane, which may be desirable for certain applications.

Because of the shape of coupling head 703, secondary connector 708 may be configured to rotate, in some cases without limitation (i.e., 360 degrees) about the axis 701 thereof. However, this embodiment may be limited in its ability to allow for pivoting of the secondary connector 708 relative to axis 701 due to the shape of the coupling head 703.

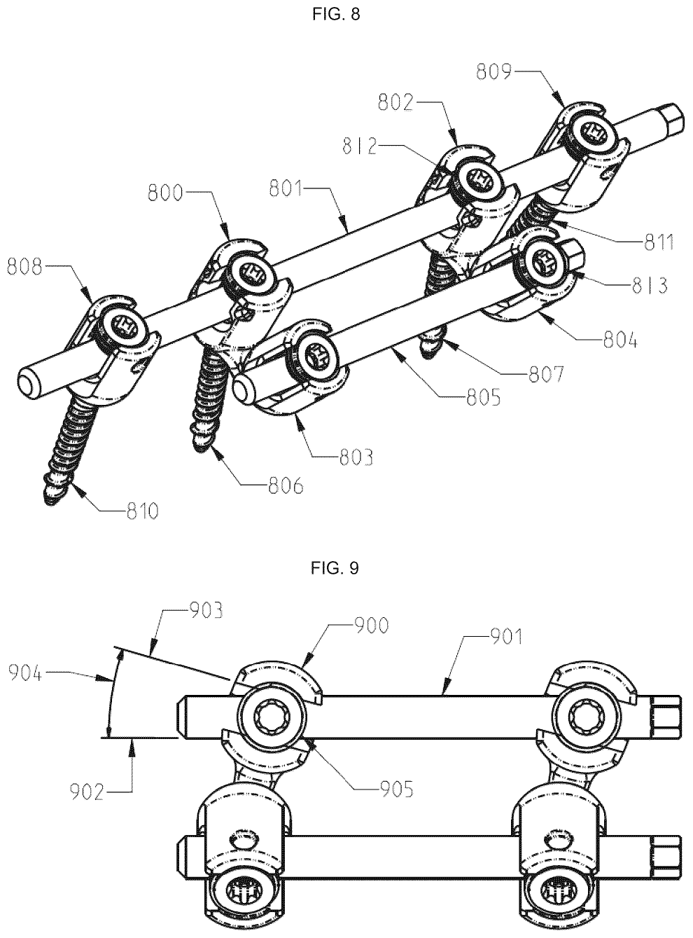

FIG. 8 is a perspective view of another spinal fixation system comprising two primary tulip connectors 800 and 802, each of which is coupled with its own secondary tulip connector 803 and 804, respectively. The primary connectors 800/802, along with auxiliary tulip connectors 808 and 809, are shown receiving a first spinal fixation rod 801. The secondary connectors 803/804 are shown receiving a second spinal fixation rod 805 that extends parallel to the first rod 801.

The first rod 801 extends through slotted openings formed in each of the auxiliary connectors 803/804, along with those of the primary connectors 800/802 and is seated and locked therein by way of a respective locking cap (see locking cap 812, for example). Each of the auxiliary connectors 803/804 and the primary connectors 800/802 further receives a corresponding bone screw. For example, auxiliary connectors 803 and 804 are shown coupled with bone screws 810 and 811, respectively. And primary connectors 800 and 802 are shown coupled with bone screws 806 and 807, respectively. Each of the various bone screws 806, 807, 810, and 811 may be configured to be polyaxially coupled to its respective connector.

Similar locking caps may be used to lock the secondary rod 805 within the slotted openings of secondary connectors 803 and 804 (see locking cap 813, for example).

FIG. 9 is a top plan view of another spinal fixation system according to additional embodiments. This figure illustrates how certain embodiments can be used to allow the primary tulip connector(s), such as connector 900 in FIG. 9, to twist or yaw in relation to the spinal rod 901 over a limited, preconfigured angle range.

As shown in FIG. 9, the axis of the rod 902 and the primary axis of one or both of the slotted openings of the primary connectors 900 may differ and therefore define an angle 904. In some embodiments, this angle 904 may be within an angle range anywhere between about ±2 degrees and ±45 degrees from the axial or neutral configuration. In some embodiments, this angle range may be more limited, which may be preferable, for example, due to the weakening of the tulip resulting from the widened portion. Thus, in some embodiments, the angle range may only allow for rotation of the rod at any angle up to about ±20 degrees. In some such embodiments, the angle range may only allow for rotation of the rod at any angle up to about ±10 degrees or, in other cases, any angle up to about ±5 degrees.

As previously explained, the facilitation of this yaw angle range may be provided by a widened portion within a slotted opening as first introduced in connection with connector 200 of FIG. 2. It should also be understood that, due to the expansion of this widened portion in both directions relative to the primary axis of the slotted opening, angle 904 may, in some embodiments, also be provided for in the opposite direction. In other words, if needed, the rod 901 may be pivoted in the opposite direction so as to form an angle with the primary axis of the slotted opening (i.e., the axis that is perpendicular, or at least substantially perpendicular, to the axis of the extending arm of the connector 900) that extends below, rather than above as shown in FIG. 9, the axis of the rod 901, due to the expansion of the slotted opening in both directions, as previously shown and described.

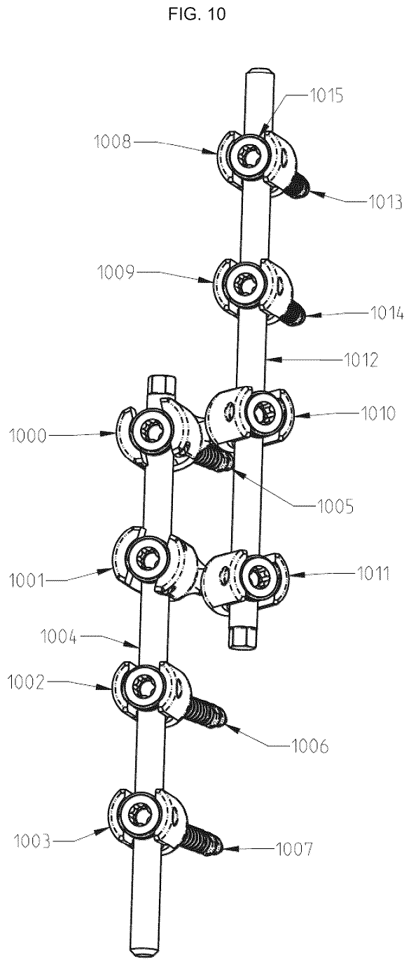

FIG. 10 is a top plan view of another spinal fixation system according to still other embodiments. In this embodiment, the primary connectors 1000/1001 may be configured to allow for adjustment of the yaw angle of the spinal rod 1004 extending therethrough, which may allow for adjustment of the various angles of the extending arms of the secondary tulip connectors 1011/1010 as needed. A second spinal rod 1012 is shown extending parallel to the first rod 1004 and is seated in secondary connectors 1010 and 1011, along with auxiliary connectors 1008 and 1009. Threaded locking caps or other suitable locking elements may be used to lock the rods 1004/1012 in place, which may also in some cases result in locking of the corresponding screws, which may be polyaxial, at a particular angle/orientation.

More particularly, one of the primary connectors 1000 has its corresponding secondary connector 1010 (more particularly, the angle of the extending arm of the secondary connector 1010) angled in a first direction (upward, from the perspective of the figure), whereas the other primary connector 1001 has its corresponding secondary connector 1011 angled in a second direction opposing that of the first direction (downward, from the perspective of the figure). This configuration may, for example, be useful in connection with fixation of the transition from the lumbar region to the narrower thoracic region.

It should be understood that this opposite angling of connectors 1000 and 1001 is the result of the ability of the rod 1004 to pivot or yaw in two opposite directions relative to the central or primary axis of their respective slotted openings, which may be provided by a widened portion of a slotted opening, as previously described. That is, connector 1000 receives spinal rod 1004 with a yaw angle in one direction that results in its extending arm going upward and connector 1001 receives spinal rod 1004 with an opposite yaw angle that results in its extending arm going downward. It should also be understood that the secondary connectors 1010 and 1011 may, if desired, have a similar widened, slotted opening to allow for rod 1012 to be similarly angled with respect to their slotted openings.

Again, each of the various connectors has a corresponding bone screw seated therein. Indeed, bone screw 1005 is shown extending from primary connector 1000, bone screws 1006 and 1007 are shown extending from auxiliary connectors 1002 and 1003, respectively. And bone screws 1013 and 1014 are shown extending from auxiliary connectors 1008 and 1009, respectively. As previously mentioned, one or more of the various connectors may further comprise a rod and/or screw locking element, such as threaded locking cap 1015.

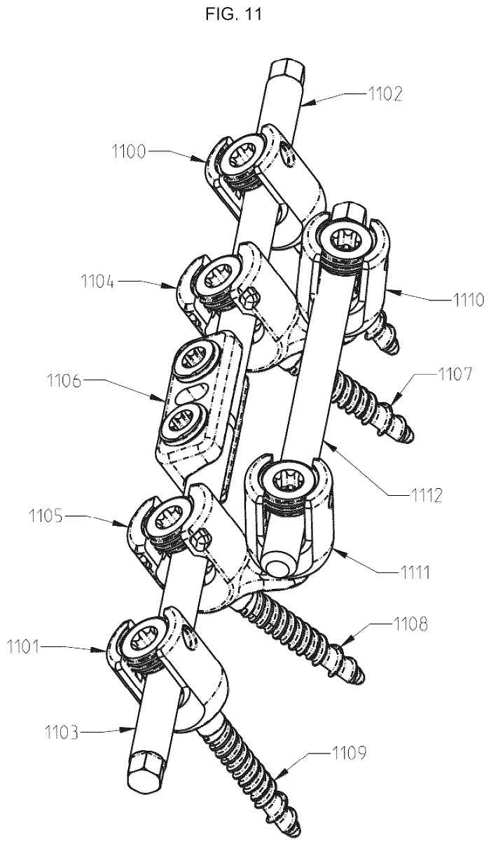

FIG. 11 is a perspective view of another spinal fixation system. The system of FIG. 11 comprises, in addition to components previously discussed, an inline connector 1106 configured to accommodate and join two aligned spinal fixation rods, namely, rods 1102 and 1103. Inline connector 1106 comprises a pair of adjacent slots, each of which is configured to receive an end portion of a respective spinal fixation rod. In addition, each slot may be configured to receive its own locking cap or other rod locking element, which may be used to engage and lock the rods 1102/1103 in place.

The other elements may be as, or similar to, those previously described. For example, each of the aforementioned aligned rods 1102 and 1103 is received by a respective primary tulip connector 1104/1105, and each of which is also coupled with a respective bone screw 1107/1108. Similarly, each of the rods 1102 and 1103 is also received by a respective auxiliary tulip connector 1100/1101, each of which is also coupled with another respective bone screw (bone screw 1109 is visible in the drawing).

In addition, a third spinal fixation rod 1112 is received by respective secondary connectors 1110 and 1111, each of which extends from an extending arm of a respective primary connector 1104/1105. This embodiment may be useful in connection with, for example, revision surgery when repairing a broken spinal rod or extending the construct to additional vertebrae without replacing the spinal rod.

FIG. 12 is a perspective view of yet another spinal fixation system. This system comprises a first pair of spinal fixation rods 1207 and 1215 that extend parallel, or at least substantially parallel, to one another using features and/or techniques previously described. However, the system further comprises a second pair of transverse spinal fixation rods 1218 and 1221 that are also parallel, or at least substantially parallel, to one another but which extend perpendicular, or at least substantially perpendicular, to the first pair of rods 1207/1215.

This feature may be accommodated by use of secondary connectors 1216, 1217, 1219, and 1220 that are rotatable on their respective coupling heads (not visible in figure). As previously explained throughout this disclosure, each of the secondary connectors may extend from a respective extending arm 1201, 1209, 1204, and 1212 that extends from a respective primary connector. Although the ability to rotate, in some cases fully, about a respective coupling head may be sufficient to accomplish the fixation depicted in FIG. 12, it should be understood that some embodiments may provide for greater adjustment flexibility.

For example, in some embodiments, one or more of the secondary tulip connectors may be, in addition to or as an alternative to being rotatable be adjustable in other ways, such as angularly pivotably upwards and/or downwards. Thus, some embodiments may be configured to provide for adjustable in roll, pitch, and/or yaw of a secondary tulip connector with respect to its respective primary tulip connector, its respective coupling head, and/or one or more other elements of the system.

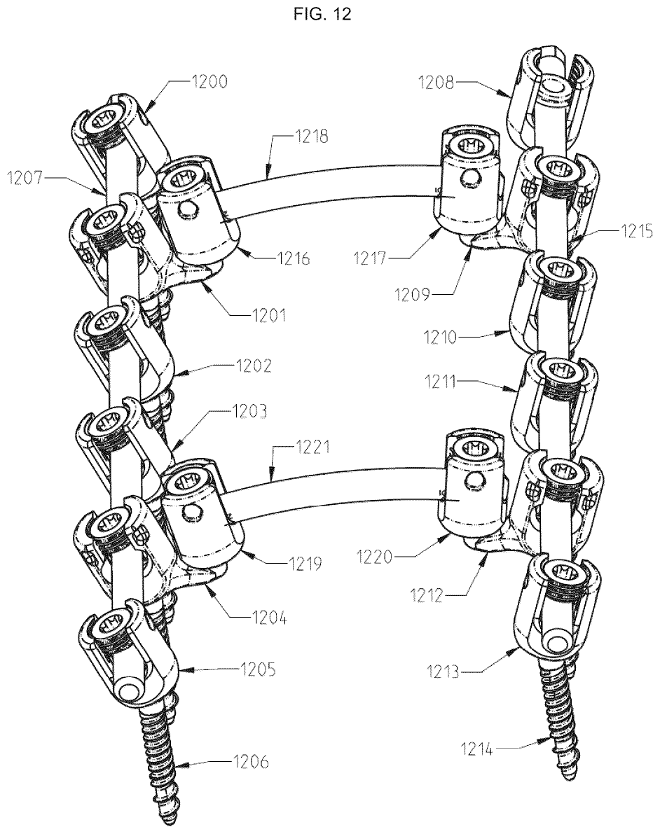

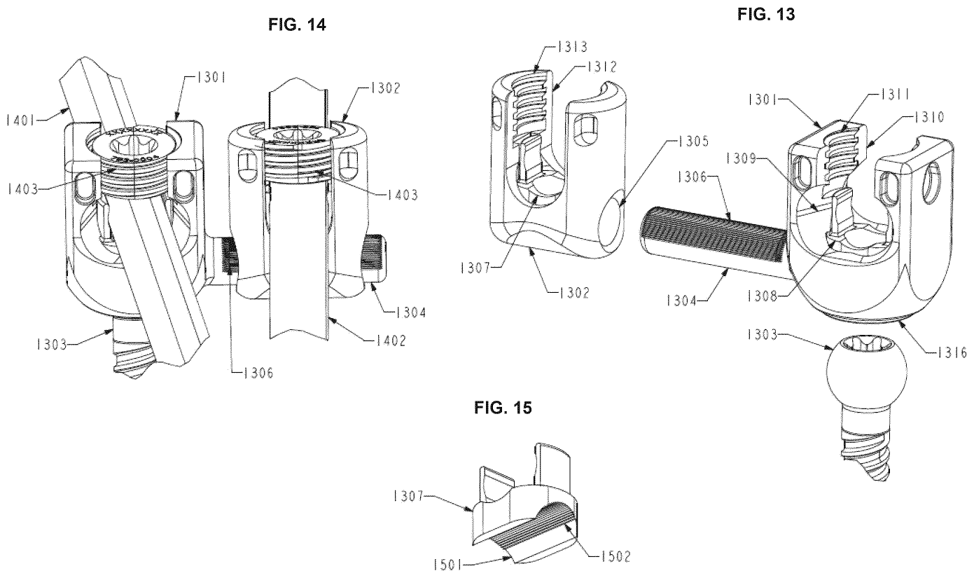

FIGS. 13 and 14 depict still another embodiment of a spinal fixation system. In the embodiment depicted in these figures, a primary connector 1301, which comprises a tulip body in the depicted embodiment, is configured to be coupled with a secondary connector 1302. As with previously described embodiments, the body of primary connector 1301 comprises a distal opening 1316 that is configured to receive the head of a bone screw 1303.

An extending arm 1304 extends integrally from the tulip body of primary connector 1301. A series of rotational locking features 1306 are formed along a portion of extending arm 1304. In the depicted embodiment, the features comprise linear, parallel grooves 1306. In the depicted embodiment, grooves 1306 are formed only along the top surface of the extending arm 1304. However, it is contemplated that, in alternative embodiments, such grooves may be formed elsewhere—such as along the bottom portion instead—or may be formed about the entire circumference of the extending arm 1304.

Similarly, although grooves are shown in the depicted embodiment, alternative embodiments are contemplated in which rotational locking features 1306 may comprise other structures that are configured to engage with one or more components of the secondary connector 1302 to facilitate locking the primary connector 1301 at a desired rotational orientation with respect to the secondary connector 1302. Examples of such alternative structures include teeth, spikes, non-linear grooves, frictional and/or surface roughened regions, and the like. As discussed below, in preferred embodiments, rotational locking features/grooves 1306 are configured to interdigitate and/or intermesh with corresponding features formed in one or more components of the secondary connector 1302.

Secondary connector/tulip 1302 includes a circular opening 1305 that is formed along its based and is configured to receive extending arm 1304 of primary connector/tulip 1301. As shown in FIG. 14, in preferred embodiments, this opening 1305 may extend all the way through the body of secondary connector 1302 from one lateral side to the opposite lateral side. However, it is contemplated that, in alternative embodiments, opening 1305 may instead comprise a blind opening that terminates within the body of secondary connector 1302.

A saddle 1307 is positioned in the secondary connector 1302. Saddle 1307 comprises grooves that interdigitate with the grooves 1306 on the extending arm 1304. Upon locking a rod 1401 within the slot or slotted opening 1312 that passes from one side adjacent to the circular opening 1305 to the opposite side, as will be discussed in greater detail below, the saddle 1307 may be pressed against the extending arm 1304, thereby locking the aforementioned two sets of interlocking grooves and locking the secondary connector 1302 in its current rotational orientation relative to the primary connector 1301.

A threaded, partial hole 1313 is formed in the secondary connector 1302, spanning across the rod slot 1312. This provides a means for threadably coupling a set screw 1403, as shown in FIG. 14. As mentioned above, threadably coupling set screw 1403 therein results in downward force on rod 1401, which, in turn, causes saddle 1307 to press against extending arm 1304. Again, the presence of grooves 1306 or other rotational locking features on extending arm 1304 thereby results in rotational locking of extending arm 1304 within hole/opening 1305.

As with secondary connector/tulip 1302, the tulip body of the primary connector 1301 includes a slot or slotted opening 1310 that passes from one side to the opposite side to allow a spinal fixation rod 1402 (see FIG. 14) to pass therethrough. Similarly, a threaded, partial hole 1311 is formed in the proximal end of the tulip body of connector 1301, which hole 1311 is configured for threadably receiving a set screw 1403.

A saddle 1308 is also positioned within the body of connector 1301. Saddle 1308 is configured to engage a rod 1401 positioned thereabove, and also to engage the head of a pedicle screw or other bone screw 1303 positioned therebelow. By threading set screw 1403, the rod 1401 engages the saddle 1308, which, in turn, engages the head of bone screw 1303. This may allow the bone screw to be polyaxial and be locked into place at any desired angular configuration relative to connector 1301.

In this embodiment, a widened portion 1309 is formed in the tulip body of connector 1301. As discussed above in connection with some other embodiments, this widened portion 1309 is configured to allow the rod 1401 to be rotated and locked into place at a number of different angles within the tulip body. For example, in some embodiments, the saddle 1308 may be partially rotatable within the tulip body, which thereby allows the rod 1401 contained therein to be rotated within the saddle to the degree allowed by the widened portion 1309. In some such embodiments, the saddle 1308 and/or rod 1401 may be rotated, for example, by 5, 10, or 15 degrees in either direction from a direction perpendicular to the axis of the extending arm 1304.

The same locking force provided for by the set screw 1403 on the rod 1401 and head of screw 1303 may also lock the rod 1401 and/or saddle 1308 in place within its current orientation. In alternative embodiments, however, no rotation may be provided for or allowed.

As alluded to above, FIG. 14 depicts the embodiment of FIG. 13 in an assembled configuration. In this configuration, the extending arm 1304 of connector 1301 passes entirely through the secondary connector 1302, as previously described. Spinal rods 1401/1402 extend through their respective slotted openings 1310/1312 of connectors 1301/1302 and set screws 1403 secure the rods 1401/1402 into these respective bodies and secure the bodies, as well as the body of the secondary connector 1302 to the extending arm 1304 of primary connector 1301. Also, in the configuration depicted in FIG. 14, the tulip body of connector 1301 is coupled to bone screw 1303, which is also locked into place by the tightening of the set screw 1403 in the aforementioned threaded, partial hole 1311 formed in the proximal end of the tulip body of connector 1301.

FIG. 15 illustrates the lower surface of saddle 1307 of secondary connector 1302. As better seen in this figure, saddle 1307 comprises a concave inner surface 1501 that is configured to engage the upper surface of extending arm 1304 of primary connector 1301. In the depicted embodiment, elongated grooves 1502 are formed along surface 1501. Grooves 1502 are configured to engage and interlock with grooves 1306 on extending arm 1304. In this manner, after being forced downward by set screw 1403, rod 1402, in turn, forces saddle 1307 against extending arm 1304, thereby interlocking the two aforementioned sets of grooves 1306/1502.

The foregoing specification has been described with reference to various embodiments and implementations. However, one of ordinary skill in the art will appreciate that various modifications and changes can be made without departing from the scope of the present disclosure. For example, various operational steps, as well as components for carrying out operational steps, may be implemented in various ways depending upon the particular application or in consideration of any number of cost functions associated with the operation of the system. Accordingly, any one or more of the steps may be deleted, modified, or combined with other steps.

Further, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced, are not to be construed as a critical, a required, or an essential feature or element.

Those having skill in the art will appreciate that many changes may be made to the details of the above-described embodiments without departing from the underlying principles of the invention. The scope of the present invention should, therefore, be determined only by the following claims.

1. A spinal connector device, comprising:
a body, comprising:
an opening formed in a distal end of the body, the opening configured to receive a portion of a fastener therein;
a slotted opening formed along a proximal portion of the body, the slotted opening configured to receive a spinal fixation rod therein;
an extending arm integrally extending from the body; and
a coupling member integrally extending from the extending arm, wherein the coupling member is configured to be received within a body of a separate spinal connector device.

2. The spinal connector device of claim 1, wherein the coupling member comprises an at least substantially spherical coupling head.

3. The spinal connector device of claim 2, wherein the coupling member is configured to mimic a pedicle screw head.

4. The spinal connector device of claim 1, wherein the coupling member defines an at least substantially cylindrical lateral surface.

5. The spinal connector device of claim 1, wherein the extending arm extends below a lower end of the body defining the opening.

6. The spinal connector device of claim 1, further comprising a second extending arm integrally extending from the body.

7. The spinal connector device of claim 6, further comprising a second coupling member integrally extending from the second extending arm, wherein the second coupling member is configured to be received within a body of a separate spinal connector device.

8. The spinal connector device of claim 7, wherein the second extending arm extends from an opposite surface of the body relative to the extending arm.

9. A spinal tulip connector system, comprising:
a first tulip body comprising a slotted proximal opening configured to receive a spinal fixation rod therein and a distal opening configured to receive a screw head therein;
a coupling head integrally coupled with the tulip body; and
a second tulip body comprising a distal opening configured to receive the coupling head therein.

10. The spinal tulip connector system of claim 9, further comprising an extending arm integrally extending from the tulip body, wherein the coupling head integrally extends from the extending arm.

11. The spinal tulip connector system of claim 10, wherein the coupling head integrally extends at an at least substantially perpendicular angle from the extending arm.

12. The spinal tulip connector system of claim 9, further comprising a bone screw comprising a bone screw head, wherein the bone screw head is configured to be received in the distal opening of the first tulip body.

13. The spinal tulip connector system of claim 12, wherein the coupling head comprises a shape and size that at least substantially matches a shape and size of the bone screw head.

14. The spinal tulip connector system of claim 9, wherein the coupling head comprises a plurality of facets configured to facilitate locking of the second tulip body at a desired orientation.

15. A spinal connector system, comprising:
a primary connector, comprising:
a body defining a slotted opening configured to receive a rod therein;
an opening formed in a lower portion of the body, the opening configured to receive a portion of a bone fastener therein;
an extending arm extending from an outer surface of the body; and
a coupling member extending from the extending arm; and
a secondary connector, comprising:
a body; and
an opening formed in a lower portion of the body, the opening configured to receive the coupling member therein to secure the primary connector to the secondary connector.

16. The spinal connector system of claim 15, wherein the primary connector comprises a tulip connector, and wherein the secondary connector comprises a tulip connector.

17. The spinal connector system of claim 15, wherein the extending arm integrally extends from the outer surface of the body.

18. The spinal connector system of claim 15, wherein the secondary connector further comprises a proximal opening positioned above the opening.

19. The spinal connector system of claim 18, wherein the secondary connector further comprises a secondary body portion laterally offset from the body, wherein the secondary body portion comprises a slotted opening configured to receive a rod therein.

20. The spinal connector system of claim 15, wherein the secondary connector is configured to be adjustable relative to the primary connector in roll, pitch, and yaw.
